SEQUENCE LISTING (14 sequences)

SEQ ID NO: 1 - DNA, 30 bp, Artificial Sequence
  Chromosome #1
tctcggctttgttttattttgttttggttt

SEQ ID NO: 2 - DNA, 30 bp, Artificial Sequence
  Chromosome Y
tacccaaactataaatatcagcctcatcgg

SEQ ID NO: 3 - DNA, 17 bp, Artificial Sequence
  SRY1 Forward
aatgggggccatgtcaa

SEQ ID NO: 4 - DNA, 19 bp, Artificial Sequence
  SRY2 Backward
tcatgagactgccaaccac

SEQ ID NO: 5 - DNA, 21 bp, Artificial Sequence
  SRY3 Forward
tcatgagactgccaaccacag

SEQ ID NO: 6 - DNA, 21 bp, Artificial Sequence
  SRY4 Backward
catgaccaccaccaccaccaa

SEQ ID NO: 7 - DNA, 21 bp, Artificial Sequence
  DNS1 Forward
atgcttggccagtgtacatag

SEQ ID NO: 8 - DNA, 22 bp, Artificial Sequence
  DNS2 Backward
tccggaaagcagccattggaga

SEQ ID NO: 9 - DNA, 23 bp, Artificial Sequence
  DNS3 Forward
gagtgcctcatctatacttacag

SEQ ID NO: 10 - DNA, 24 bp, Artificial Sequence
  DNS4 Backward
tctagttcattgttgattagttgc

SEQ ID NO: 11 - DNA, 21 bp, Artificial Sequence
  PYF Forward
aatccaccatacctcatggac

SEQ ID NO: 12 - DNA, 20 bp, Artificial Sequence
  PYR Backward
tttctcctgtatcctcctgc

SEQ ID NO: 13 - DNA, 21 bp, Artificial Sequence
  PCF Forward
gttgcactttcacggacgcag

SEQ ID NO: 14 - DNA, 21 bp, Artificial Sequence
  PCR Backward
ctagcccattgctcgccatag

(19) United States
(12) Patent Application Publication
(10) Pub. No.: US 2010/0167336 A1
(43) Pub. Date: Jul. 1, 2010

(54) PARAMETER FOR X- AND Y- CHROMOSOME BEARING SPERM SORTING WITH HIGH DEGREE OF PURITY

(75) Inventors: Jung Ho Son, Chungcheongnamdo (KR); Jay Hong Whang, Seoul (KR); Young Seok Gang, Seoul (KR)

Correspondence Address: EDWARDS ANGELL PALMER & DODGE LLP, P.O. BOX 55874, BOSTON, MA, 02205, US

(73) Assignees: NOAH BIOTECH, INC., Cheonahn (KR); XYZ BIO INC., Davis, CA (US)

(21) Appl. No.: 12/676,217

(22) PCT Filed: Sep. 4, 2008
(86) PCT No.: PCT/KR08/05211

§ 371 Date: Mar. 3, 2010

(30) Foreign Application Priority Data
Sep. 4, 2007 (KR) 10-2007-0089269
Apr. 9, 2008 (KR) 10-2008-0032903

(51) Int. Cl. C12Q 1/02

(52) U.S. Cl. 435/29

(57) ABSTRACT

Current invention is about the sex-specific separation of sperm in high purity. For more details, this invention is about the methods for improving the separation efficiency of sperm by removing factors deteriorating separation efficiency by utilizing several parameters and adjusting few conditions for the purpose of separating sperm into X-chromosome bearing and Y-chromosome bearing groups in high purity. Current invention, for the purpose of separating X-chromosome bearing and Y-chromosome bearing sperm based on physiological characteristics, utilize the difference of sperm's nucleus width as a parameter to recognize the difference in DNA content between X and Y chromosome. Current invention is about the sex-specific separation of sperm in high purity. This invention, unlike any known conventional methods, requires neither special conditions nor special treatments, can be conducted in a more realistic environment and, as a results, sex-specific sorting of sperm can be conducted without losing viability or motility. Therefore, the limitation conferred on sorting efficiency by the motility can be overcome by our invention.

### TECHNICAL FIELD

The present invention relates to a new sperm sexing method for removing factors that affect sperm sorting purity, and for the control of several parameters that enhances sperm sorting efficiency thereby having high purity of X- and Y-chromosome bearing sperm population.

### BACKGROUND ART

For the production of mammals and fish embryo in vivo or in vitro and artificial insemination, necessity of selection of X- or Y-chromosome bearing spermatozoa has been increased in the aspects of animal production efficiency of predetermined sex, management and commercial approach. So far, many submitted patents for sperm selection method were based on sperm mass, concentration and size wherein sperm swim through glass bead or columns and centrifugation (U.S. Pat. No. 5,135,759, U.S. Pat. No. 4,474,875, U.S. Pat. No. 5,514,537, U.S. Pat. No. 4,605,558, U.S. Pat. No. 4,009,260), but the high purity sperm population can not be obtained by the methods described above.

Although sperm sorting method using flow cytometer has been introduced recently, it is based on sperm volume, size, mass and DNA content (PCT/US2001/15150), and the methodology was not considered sperm physiology of different species, and submitted patent's sorting event, purity and fertility following insemination using sorted sperm were questionable. In sorting sperm by flow cytometer, many researchers used only forward scatter (FSC) and side scatter (SSC) parameters to identify the X- and Y-sperm population, but using only two parameters described above have limitation, resulting in low purity.

The sperm sorting methods mentioned above have several prerequisites which make the methods impractical for fertilization as sorted sperm are not alive any more. For example, sperm should be fixed with formalin and their tails being removed by ultrasonification for better sperm orientation while sorting. To obtain the each sperm's exact size, it assumes that tails need to be removed, sperm should not be moving while being sorted, and sperm's alignment from the sperm that pass through sample line should be the same. Although methodology described above adopt the improved nozzle or stream stabilizing technology, live sperm, in reality, are motile and therefore it is hard to align all sperm's moving direction uniform, resulting in reduced number of sorting events and also motility was overlooked after sorting. On the other hand, another sperm sorting method is DNA amount evaluation in sperm nucleus in measuring the emitted fluorescence amount as a sorting parameter from the dyed cell. Problem being, it is incapable of measuring DNA amount by analytical chemistry in nucleus and also by the fluorescence intensity difference from forward scatter (FSC) and side scatter (SSC) parameters due to no distinct differentiation in high ratio of mixed cell population on plot by those two parameters, resulting in low sorting purity. Method describe above merely speculate as high purity and there is no system to confirm or to check the purity.

We would like to introduce the former method that use flow cytometer, briefly. Traditionally, FSC is able to measure the sperm size (or height) and SSC is able to measure the granule density of sperm head membrane. Because depending on sperm's lying position, cell density data even from same sperm showed differently and thus practically, SSC measures merely total cell volume and therefore sorting purity is lowered. It is merely theoretically possible that SSC is able to differentiate the cell, if cell moves same direction, same alignment angle and has no motility. If cells are live and have motility, it is hard to expect high degree of sorted sperm's purity. It showed the limitation that FSC is only able to identify the cell difference under the special condition and assumption such as cell should be fixed with formalin and tail should be removed from the head. Because the concept of separation method can be made at 0° (FSC) or 90° (SSC) photo multiplier tube on flat sperm head surface, it has been also done under the assumption that cells are in same angle at the time of analysis and thus cells are in different angle need to be removed, thereby possible number of separated cell events are reduced. To satisfy with the assumption and condition, many attempts were done to minimize the irregular part by encapsulation and ameliorate the nozzle for cell mobilization. But cell alignment could not be the same even by special nozzle, therefore limitation still exist.

In analyzing cell volume by encapsulation (dropping) of the sperm, whole sperm head including nucleus, chromatin, acrosome and cell membrane are measured, but in fact, volume measurement are included mid piece and head. Thus, identification is done by measuring the difference of whole capsule volume. Although complementary application of SSC such as side scatter beam's density difference of DNA amount in sperm in measuring the whole sperm volume used, sorting accuracy is still very low. Furthermore, because SSC parameter is not enough to identify the cell, emission difference of fluorescence intensity from DNA is utilized to analyze the whole capsule (in case of fluorescence intensity is the indicator per cell), after dying the DNA with fluorescence to measure the DNA amount. This method has advantageous effect compare to the method that using only FSC and SSC parameters, but it still has several problems mentioned earlier and also measures merely whole volume.

In summary, first, the sperm volume is identified using histogram obtained by FSC and SSC and second, the sperm DNA amount difference is measured by fluorescence intensity difference using fluorescence dye.

Laser and mercury ark lamp are generally used for light sources in flow cytometry. Mercury ark lamp has an advantage in easiness of use and low cost of installation and maintenance but due to the difficult of making stimulated light emission, laser is used. Argon laser (blue laser; 488 nm) or helium-neon laser (red laser; 633 nM) are used for general purpose and UV laser (355 nM) is used for the analysis of fluorescence dye such as Hoechst 33342. FSC and SSC data can be obtained by blue and red laser so UV laser doesn't need for that purpose.

### DISCLOSURE

### Technical Problem

Objective of present invention is to overcome the problems of former method described above and to improve sorted sperm's purity remarkably in utilizing new parameters that able to identify the X- and Y-chromosome bearing spermatozoa from mixed population. In order to do the high purity sperm sorting, this invention also includes optimum conditions.

### Technical Solution

To achieve the objective, DNA width difference in nucleus of sperm head used as a sorting parameter in recognizing DNA amount difference from X- and Y-chromosome bearing spermatozoa. DNA width can be recognized by measuring the passing time of dyed DNA's (in nucleolus) beginning to end that pass through laser. Also, the Area/Height value obtained from signals as nucleus pass through the laser is utilized as newly defined separation parameter on width of nucleus.

More specifically, sperm sorting procedure includes a) semen collection step from male animal; b) sperm sample preparation step wherein semen is diluted with extender; c) semen evaluation step based on sperm physiology; d) cell identification step by grouping; e) sorting step of X- and Y-chromosome bearing spermatozoa in which nucleus width difference is used as a sorting parameter. Nucleus width difference is obtained by measuring the passing time at which beginning and end of sperm nucleus passing through the UV laser. Also, sperm can be sorted based on the width of nucleus which is analyzed by dividing the area with height of laser signal. Furthermore, utilizing the combination of FSC and SSC signals and the total fluorescence intensity of nucleus as separation parameters enables the sperm sorting with high degree of purity.

### ADVANTAGEOUS EFFECTS

This invention dramatically enhances the sorting purity by adopting the new parameters in evaluating the X- and Y-chromosome bearing sperm. Also, it enhances the sorting purity by maintaining the buffer pH at a constant level. Constant pH also keeps the sperm alive and restrains sperm motility selectively.

Besides, because present invention performs the sperm sorting in natural conditions and do not requires impractical treatments which conventional invention does; our invention is practical for the purpose of sperm application after sorting. It also overcomes the limitation of live sperm sorting that pass through the sample line with various orientations of angles, thereby increases the number of sorted sperm (efficiency, yield) remarkably.

### DESCRIPTION OF DRAWINGS

FIG. 1 shows a schematic outline of the concept to explain the apparatus for flow cytometry, along with the sorting mechanism as an example of present invention.

FIG. 2 shows the different shape of sample line injection chamber between conventional methods and present invention. In case of present invention, it is shown that the sample intake speed can be increased and thus elevates the event number.

FIG. 3 shows how the viability and motility of sperm can be preserved while cells are moving through the sample line as the friction to the wall is minimized with coated inner tube surface.

FIG. 4 is a diagram to explain the concept of sorting parameters wherein width of sperm nucleus (DNA) is measured by time interval and is subsequently utilized as a sorting parameter.

FIG. 5 is a diagram to compare the measurement region of present invention which use width of nucleus and the conventional method which use whole volume.

FIG. 6 is a comparison of a conventional method which use whole fluorescence intensity as a one parameter after dying the sperm nucleus and based on sperm volume (under the specific experimental condition with aligned sperm in which sperm tail was removed) with the present invention which use nucleus width as a parameter in natural conditions.

FIG. 7 is a diagram to explain a new sorting parameter using area/height wavy pattern that generated by laser.

FIG. 8 is a dot plot created from sperm with intact tail and sperm lay in various angles during the passage as parameter explained in FIG. 7.

FIG. 9 is a diagram to make understand of present invention by showing sperm's orientation angle and the values of sorting parameter on FIG. 8.

FIG. 10 is a dot plot diagram showing sperm sex-sorting by the distribution of sperm on plot.

FIG. 11 is a diagram showing the purity evaluation of sex-sorted sperm by fluorescence in situ hybridization (FISH) method and results.

FIG. 12 is a diagram showing how purity evaluation of sex-sorted sperm by polymerase chain reaction (PCR) method and results.

### BEST MODE

From now on, more detailed information on present invention will be given. However, for a clear description of subject matters of the invention, detailed descriptions of associated well-known functions and configuration will be omitted. For example, because general information of flow cytometer, concepts of FSC and technical operation are already well known to the skilled person and also well explained in articles, it would be safely ignored.

This present invention is regard to high purity sperm sorting wherein X- or Y-chromosome bearing spermatozoa is mixed in 50:50 ratios in naturally. Generally, application of conventional methods have been limited to the sperm of mammalian species, however, this invention would be applied to fishes and other species.

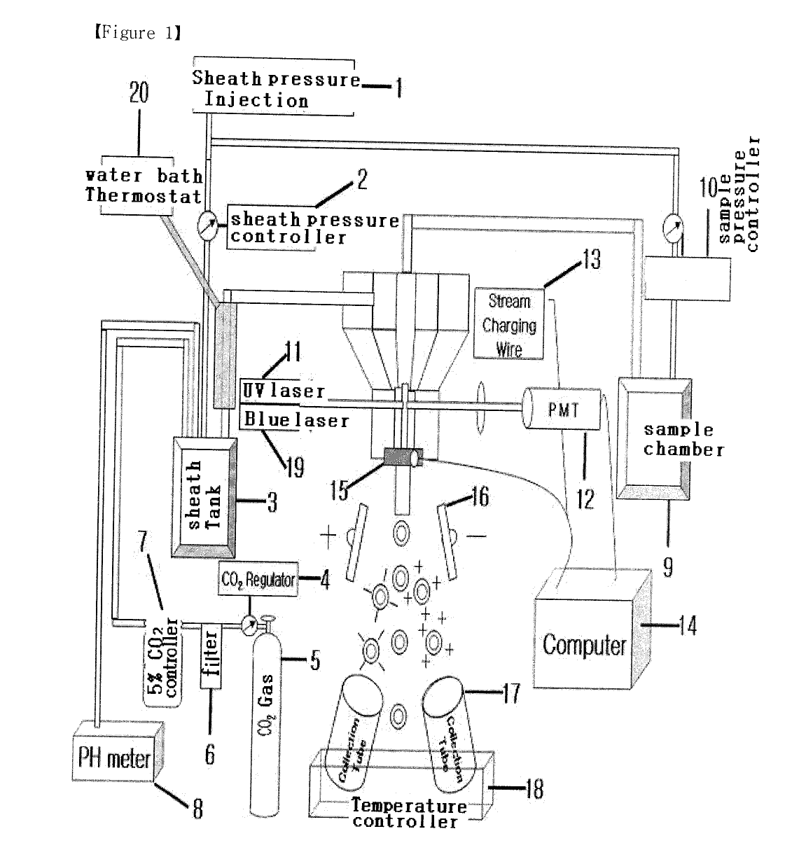

In present invention, it is desirable to use semen diluent formulated for the species specific. For the purpose of semen dilution is suppress metabolism, prevent overpopulation and cold shock of sperm, and also elevate the available amount of semen and sperm viability. During sperm sorting process, appropriate sheath buffer should be used to preserve the sperm motility and viability. Examples are shown in FIGS. 1, 2 and 3. If necessary, motility enhancer, motility suppressor, or antibiotics can be added to the sheath buffer. During sperm sorting, the accuracy and sorting event will be affected as motile sperm pass through the flow cell and DNA excitation by laser is not consistently analyzed. In present invention, we also employed the method which is put sperm into trance temporarily in a specific temperature zone to solve the problems and to elevate the sorting accuracy. It is recommended to maintain the temperature at 4° C.˜37° C. during the sorting procedure. The sperm viability may drop as temperature goes down below 4° C. and it will be shortened if kept at above 37° C. due to motility-related metabolism increase. More specifically, it is desirable to maintain the temperature range as 4˜7° C. for cow, 5˜17° C. for pig and 33˜37° C. for mouse during sorting processes.

FIG. 1 is an example of this invention, showing a diagram, including the system of flow cytometer and the mechanism of sorting process. Obtained sperm are diluted with extender specially designed for each animal species to the concentration of 1 million per ml and characterized into two groups on the flow cytometer, X-chromosome containing and Y-chromosome containing, by analyzing fluorescence emitted from illuminated sperm in the from of FSC and SSC. Subsequently, signals from each sperm were displayed as spots on the computer monitor and finally sorted by applying charge of 5000˜6000 volt (16). Emitted light signals recognized by photomultiplier were translated into electric signal, and amplified and, then, saved in computer. Especially, applying the voltage of 200˜300 was appropriate to create gatings as voltage of this range can generate signals readily amplified. In another word, sperm can be successfully grouped into X-chromosome containing sperm and Y-chromosome containing sperm. Applying 250˜300 volt is also appropriate for the gating to remove portions that affect the separation accuracy. In this invention, the term ‘gating’ means assigning and selecting individual signals into a specific group based on several standards. Meanwhile, using SSC and/or FSC as a sole parameter to analyze the size of whole sperm head may not bring a good result for the separation, as the portion of X- Y-mixed will be enlarged.

The sample is continuously supplied from the sperm sample chamber (10) by using extender. The speed of flow can be adjusted depend on the condition of sample by manipulating the sample pressure controller (10). In this invention, separation efficiency can be elevated as hidden factors deteriorating the separation accuracy are eliminated by sophisticated gating steps. Inside the flow cytometer, sperm are sent through ducts system filled with sheath solution. The high pressure of sheath solution is generated by a sheath pressure generator (1) and is also precisely controlled by a sheath pressure controller (2). The optimal pressure to minimize sperm damage is about 20 psi. As sperm move through the inside of sheath solution-filled duct system, damage to the sperm, especially on the membrane and tail, can occur. In this invention, the viability and motility of sperm were maintained at high level as the damage to sperm was minimized by coating the inside of duct with mineral oil or silicon. The system of sample line is described in FIG. 3. Although the sperm tail was taken off in conventional methods, in current invention, sperm tail is not removed. As a result, sperm are not severely damaged and their viability are well-maintained.

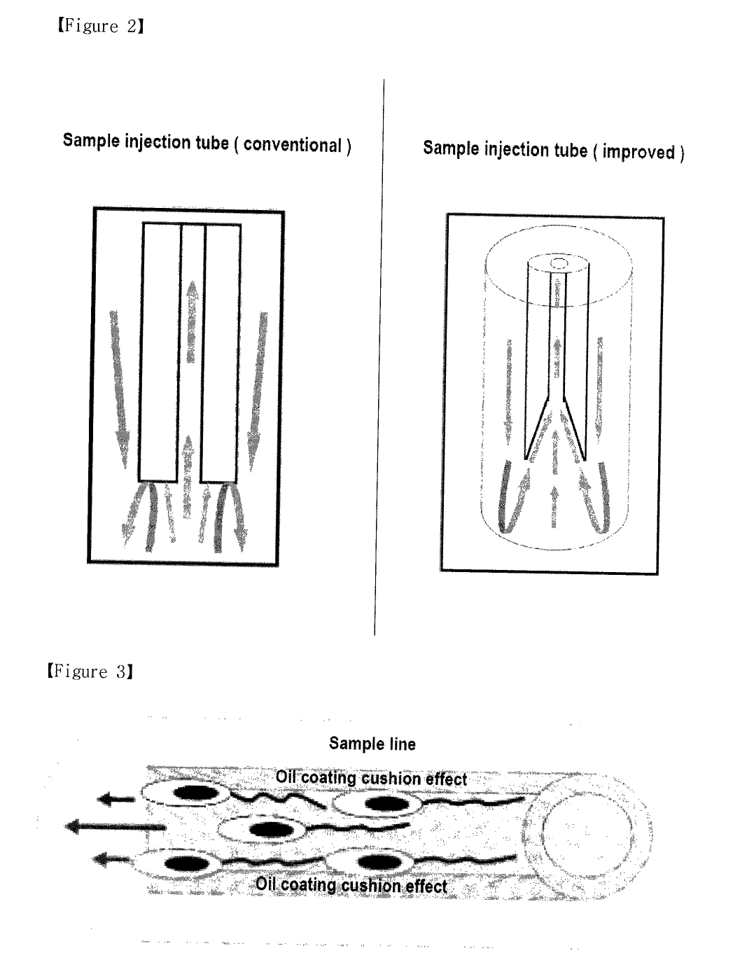

Stress to the sperm can be reduced and their viability increased by modifying the buffer solution in a species-specific manner. Maintaining the temperatures at optimal level by controlling thermostats placed in several locations, such as the sample chamber (9), the water bath (20) connected to sheath tank (3), and the collection tube (17) collecting sorted sperm can elevate sperm viability and also the separation efficiency. As mentioned previously, the optimal temperature to be maintained is from 4° C. to 37° C. In addition, the shape of injection tube at working is described in FIG. 2. By using the sample line with an entrance shaped like a letter ‘V’ sample can be taken into the machine much faster and the separation efficiency can be increased. Compared to the conventional models, the area of entrance of our invention is shaped like a letter ‘V’ and, as a result, a continuous and uniform supplementation of sample is allowed and aggregation of sperm is prevented. In addition, V shaped entrance can contribute to the lowering of damage in sperm sample

In this invention, for the purpose of elevating sperm's viability, pH fluctuation during the process of sperm sorting was controlled to the minimum level. Controller of CO₂ gas supply was located at sheath tank (3) which contains sheath buffer (4, 5, 6, 7, 8). During the process of sorting in flow cytometer, pH can fluctuate as the sodium bicarbonate-containing buffer solution is exposed to the air and, as a result, sperm can lose their viability. In this invention, pH was continuously measured using a pH meter and the CO₂ (5%) supply was regulated to stabilize solution's pH and to elevate sperm's viability. Although it can be variable depend on species, the pH range of 6.8˜7.4 are appropriate. Generally, pH 7.2˜7.4 is more appropriate and pH higher than 7.4 can cause abnormality as a result of increased metabolism. In case of pig sperm, pH 6.8 is appropriate and, therefore, it is recommended adjust the pH to 6.8˜7.4.

From now on, explanation on the separation parameters which are the most important part of our invention will be given.

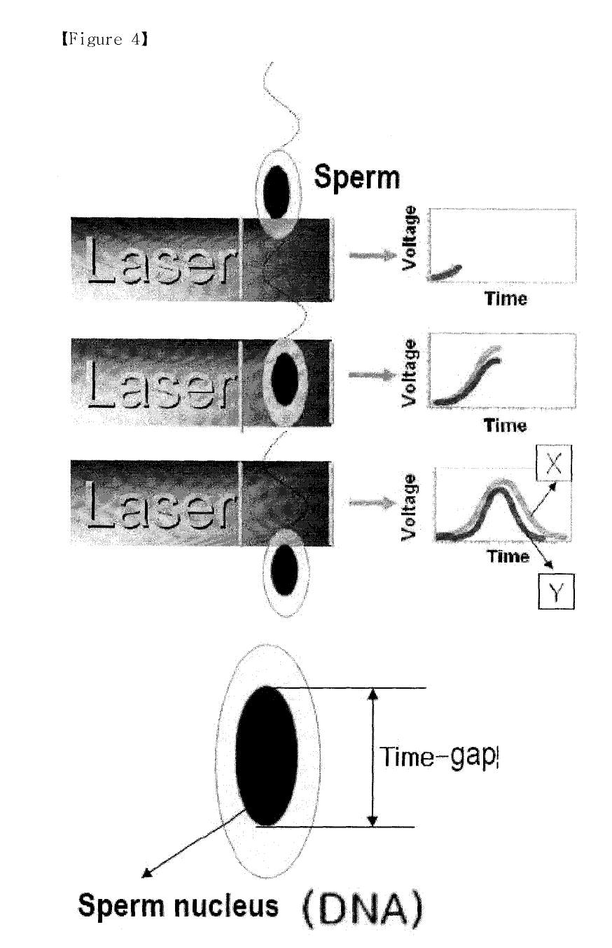

In this invention, signals generated when sperm are illuminated with UV laser (11), were collected and utilized as a parameter for the analysis on computer (14) to create a population diagram for gating. FIG. 4 shows the signals generated from sperm exited by the UV laser illumination in a time-course. The concept of width of sperm nucleus can be introduced at this point. The width of sperm nucleus (or DNA) which can be used as a important difference between X- and Y-sperm, are analyzed by the time taken for the nucleus to pass the UV laser. The gap between two time points, first moment when the edge of nucleus begin to enter the area of laser and the second moment when the nucleus completely leave the laser, are measured and converted into nucleus width. By doing this analysis, it was confirmed that the nucleus of X-sperm is actually wider than that of Y-sperm. This means that region available for sorting become wider and that the number of events and the accuracy of sorting can be improved. Additional information on the width of nucleus will be given. As can be seen in FIG. 4, the width of nucleus can be measured by using the time during which the nucleus is illuminated with laser beam. In another word, nucleus size can be expected to be bigger as the time taken to illuminate nucleus get longer. Sperm can be in many different orientations when they moving down. Some may be in a complete vertical orientation while others are in different orientations.

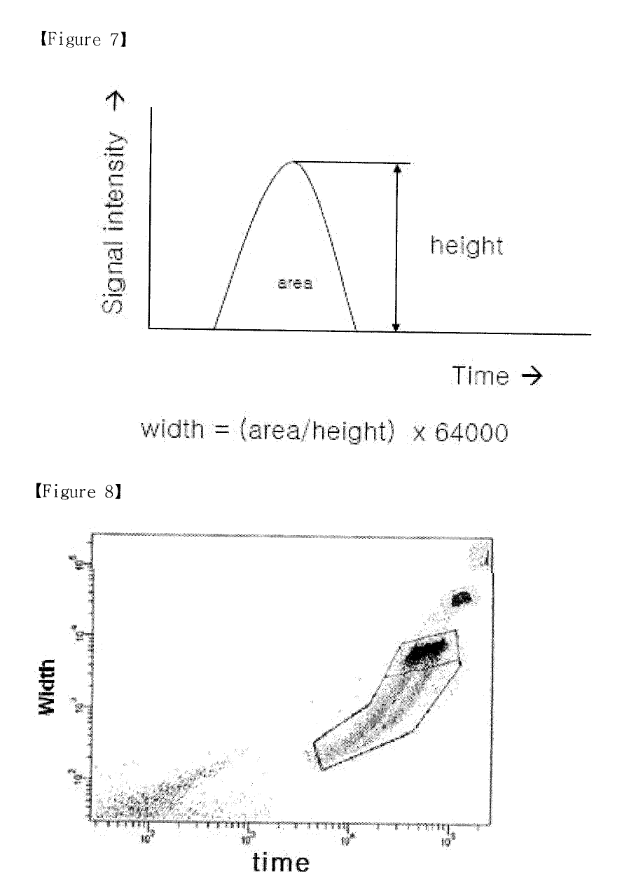

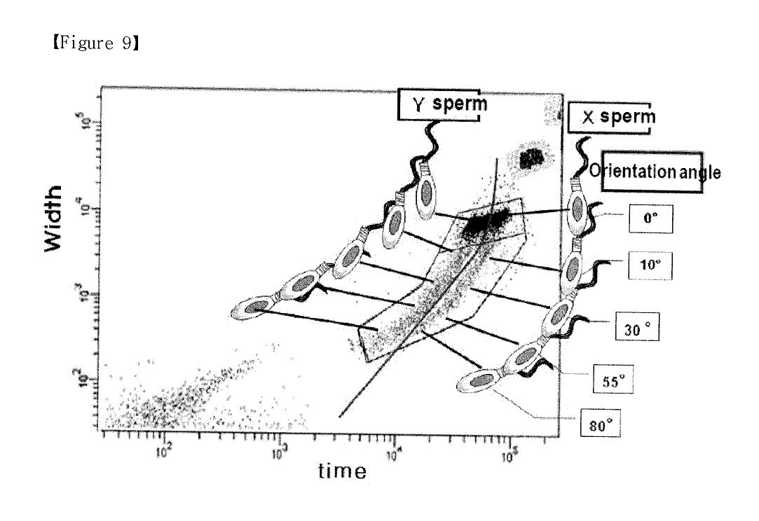

As can be seen in FIG. 4, for nucleus of sperm moving down in a complete vertical orientation, maximum nucleus diameter can be equal to nucleus width and for nucleus coming down in a 90° slope, minimum nucleus diameter can be equal to the nucleus width. Comparing the width of nucleus of X- and Y-sperm can be meaningful only if most sperm move down in same orientation. However, if they move down in random orientation, it is necessary to employ additional parameters on the width of nucleus. As mentioned in FIG. 7, difference in detected signal is generated due to the difference of the time taken for the nucleus to pass through. By using this, a new parameter can be obtained by dividing wave area with the height of wave. This value can be used as an additional sorting parameter. The concept of nucleus width is about how to define the nucleus based on the difference of physiological characteristics. Firstly, the time taken for a nucleus to be illuminated by laser beam when sperm passing through the flow cytometer can be regarded as nucleus width. Secondly, the value obtained by dividing the area with the height of the wave signal, generated during the contact of nucleus and laser beam can regarded as a nucleus width (“regarded as nucleus width” means that the difference of characteristics of nucleus are defined based on standpoints described above). Although employing just one of the standpoints mentioned above can lead to a chance of successful sorting, as can be seen in FIG. 9, if both are applied, sperms coming down in a slope can be sorted to a certain degree. In this figure the whole concept of sorting mechanism was explained comprehensively although more detailed information will be given in a separate figure.

Generally, UV laser has been used to detect nucleus stained with fluorescence chemicals. The measurement can be more accurate if used with higher powered laser, although this can cause damage to sperm. This is also a problem to be solved for the successful sperm sorting. UV powered by 20 mW does not cause severe problem to sperm, but is not strong enough to differentiate DNA contents of X- and Y-sperm based on their emitted fluorescence. Therefore, generally, UV powered by 40 mW or above has been used for the sorting purposes. However, blue laser (488 nm) can replace UV laser as the fluorescence can be generated from the sample by illuminating either one of two light sources. Hoechest 33342 generates fluorescence after being illuminated with blue laser and also significant difference of fluorescence can be detected. In this invention, it is recommended to use UV laser and blue laser simultaneously. Emitting of fluorescence can be analyzed with blue laser and sperm nucleus width can be analyzed with UV laser, but is not limited to these. It is desirable to use laser of low watt for the purpose of maintaining sperm activity and minimizing any damage.

It is recommended to use both laser beams simultaneously. Blue laser not only measures fluorescence emitting but also can measure FSC and SSC. Generally FSC emitted from the object illuminated with blue laser can be used to give data on the size and shape of whole cell. On the other hand, SSC generated in a similar way, can be used to analyze the granulation of cytoplasm (density). Since Y-chromosome containing sperm are generally smaller and contain DNA of higher density within the nucleus, these characteristics can be utilized for the purpose of sorting. However, as mentioned previously, since the difference is later small, sperm can not be sorted accurately with this parameter. Although solely depending on the density and size from SSC and FSC as parameters for the plotting and gating can be an inefficient method, however, these parameters can be very useful for better and accurate sorting if used as additional parameters.

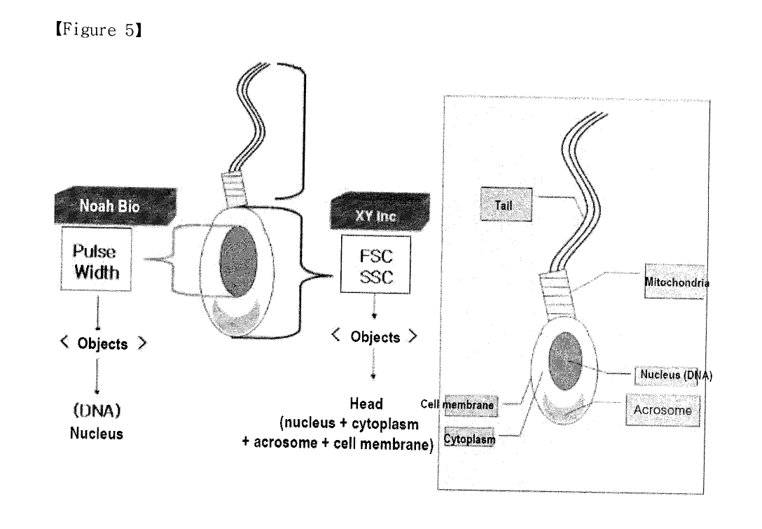

FIG. 5 shows detected portion of the nucleus width in this invention and detected portion by SSC and FSC. Although SSC detect the granulation and pigmentation of the cytoplasm of the cell but actually this data can be interpreted as the volume of the sperm head. Since the density of the cytoplasm of sperm head is not homogeneous, what really can be measured is the volume of sperm head. Also other problems can be expected due to the difficulty of standardization of X- and Y-sperm differentiation caused by increased variation of sperm head size and irregular detection range.

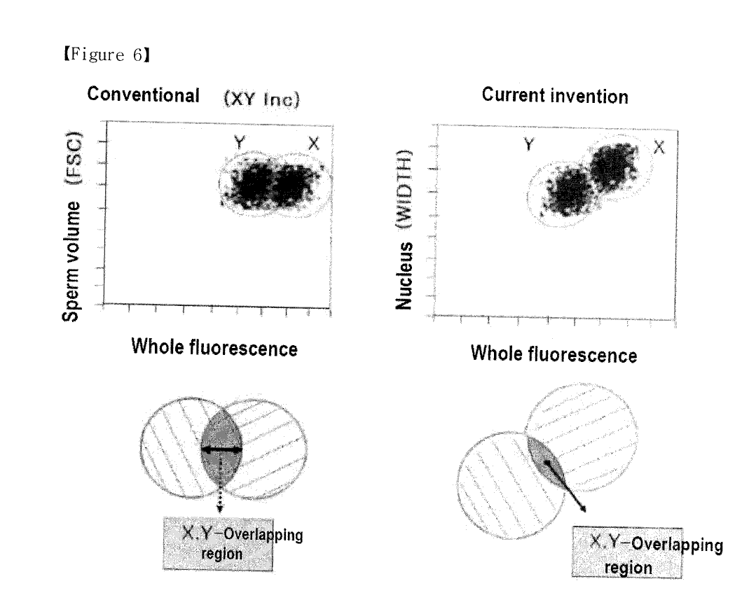

In FIG. 6, difference between methods of this invention and methods of XY INC which is mentioned as a conventional technique was explained. In conventional methods, the whole fluorescence emitted from nucleus of sperm or the size of sperm was used as a parameter. In this invention, instead of sperm [symbol] size, the fluorescence emitted from sperm nucleus or the width of nucleus was employed as a parameter. In older method, to be more faithful to the conditions of the XY INC, tail was removed from the sperm by using centrifugation and ultrasonication, sperm were kept in lower temperature to minimize motility and to unify sperm's orientation, condition of older method were followed faithfully. Nonetheless, effectiveness of the sperm size as a sorting parameter is much less effective than what it should be. In contrast, staining nucleus with fluorescence dye and analyzing the whole fluorescence as a parameter showed a better resolution for the differentiation. In the left figure, X and Y sperm were differentiated by utilizing emitted fluorescence. However, the difference were not noticeable when sperm are compared using their volume. In this case, the difference may can be noticed if PMT voltage is controlled by very experienced person, however, generally we can conclude that it is not a good parameter for the purpose of sperm sexing. In this invention, since the width of nucleus shows significant difference between sperm of two sex, it can be employed as a parameter for sorting. For better understanding, we can use the diagram of X- and Y-sperm. When the parameter of our invention is employed, area of mixed sex shrinks and sperm of higher purity can be obtained. This indicate that our invention is much more effectiveness for the purpose of sorting sperm, although, purity can be even higher, if the parameter of sperm volume employed simultaneously.

Also a time-course change in wave shape is generated when the nucleus pass through the laser beam. Information on change in wave shape can be collected and analyzed to define the width of nucleus of sperm head. In another words, the wave area and wave height of the wave can be utilized as another parameter. As explained in FIG. 7, the area under the wave and the height of wave can form another parameter, which is the value obtained by dividing wave area with the wave height. Advantage of this parameter is that it analyzes the nucleus of sperm and often allows differentiation of cells known to be difficult to sort.

And since the shape of sperm is not always ball-like, but it can be variable in a species-specific manner, data can be influenced differently by the movement and positioning of sperm and also by species-specific characteristics. Therefore, attempt to sort sperm based on obtained data without considering species specificity, can lead to a serious misjudgment, which means sperm of mixed sex can be included in the gating range. In another words, miss-analysis can occur during the sorting process if sperm are not oriented rightly. Data can be even more obscure if live sperm are analyzed without their tail being removed. FIG. 8 shows a dot blot obtained from analysis of live sperm with intact tail sorted with our protocol and a normal nozzle. Unlike cases in FIG. 6, it can be seen that sperm distribution can be variable of diverse manner, if nucleus width obtained in a function of consumed time, along with wave shape generated by the difference of width are applied simultaneously. In FIG. 7, it is shown that regularly grouping and meaningful distribution without application of additional prerequisites.

On the other hand, in conventional methods, generally UV laser have been employed for the analysis of whole fluorescence. Although, in older methods, the main reason to employ UV laser was to detect the difference in fluorescence, in our invention, UV laser was employed to obtain parameters necessary for the measurement of nucleus width and blue laser was employed to analyze the whole fluorescence. The reason is that blue laser can replace UV laser for the measurement of the side scattering C, forward scattering C, and whole fluorescence without seriously damaging sperm. Therefore it is desirable to choose blue laser to measure the whole fluorescence.

FIG. 9 explains cases of sorting sperms positioned un-uniformly. When sperm are positioned askew, the diameter calculated from the time taken for sperm nucleus to pass the detection range will be decreased as shown in the figure. However, at the same time, the shape of a wave generated when sperm pass through the range of laser field will also be changed. If these changes are expressed along with the nucleus diameter data defined with the area and wave height of sperm nucleus, the distribution of X- and Y-sperm can be visualized. In our invention, even sperm oriented askew can be included into the range, thus, the loss of sperm can be minimized. If sperm passing uprightly are characterized, the time, taken for X-sperm to pass through, become larger than that of Y-sperm. In terms of shape of wave, X-sperm is little bit bigger and also the difference can be seen on blue area of the FIG. 9. If sperm passing askew are characterized, sperm of larger nucleus, passing askew, can be confused with sperm of smaller nucleus, passing upright the field. However, in this case, differences of wave shapes still exist and this difference can be utilized to characterize the width of sperm and to differentiate X- and Y-sperm. In contrast, if the shapes of wave are not clearly different, the data of consumed time to pass through can be utilized for differentiation. Therefore, the important advantage of our invention is that sperm passing askew can also be characterized and included in the gating range. Another advantage of this invention is that the detection of these two parameters, consumption of time to pass through and the characteristic wave shape can be performed simultaneously.

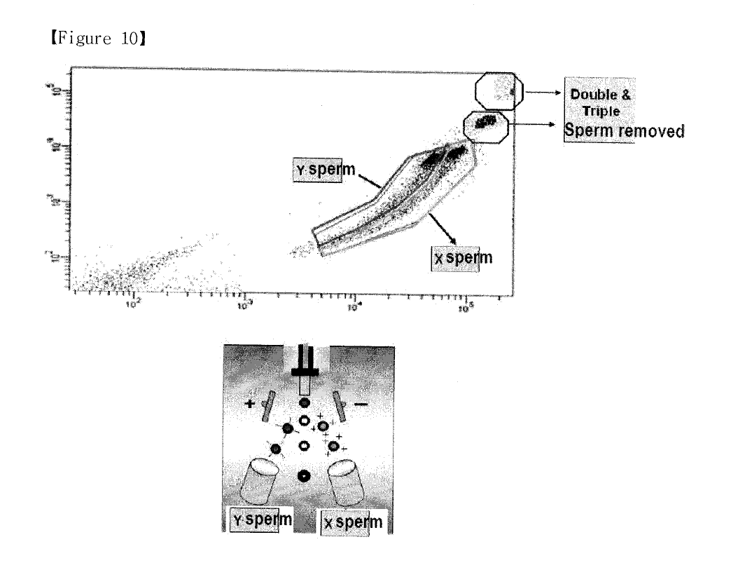

In this invention, gating range was decided to avoid the contamination by sperm in X-, Y-mixed range as much as possible. As shown in FIG. 10, signals indicating unusually wider nucleus, due to the aggregation of two or three sperm heads, was not included in gating range. As the range of gating is ordered in computer, sperm will be separated into either X-sperm, Y-sperm, or waste when sperm pass through high voltage activated plates.

From now on, factors affecting the gating of sperm and few interesting applications of our invention will be briefly mentioned.

Specific group identification is possible by photo multiplier tube (PMT) adjustment. It is recommend to have removing step by PMT adjustment to 200˜350 volt, more desirably, within a range of 250˜300 volt in differentiating the aggregated sperm in either dead or live. To further increase the sorting efficiency, older methods can be applied to our protocol. In older methods, increasing the sorting speed generally deteriorated sorting purity. Increasing sorting speed affects sorting accuracy due to the crossing over the each sperm populations. In conventional method, differentiation of sperm populations on dot plot image are not obscure but present invention can differentiate the sperm due to the certain phenomenon shown on dot plot image even if aggregated sperm and sperm lie in different angle due to motile. Therefore, present invention does not affected by increasing sorting speed and changes of event number, and has huge advantages.

Among animal's sperm, the one of mouse has a hooked shape. In this case, sorting was not possible when sorting was attempted with older methods and that sorting can be successfully conducted only when sperm are sorted with the application of parameter of the width of nucleus. In another word, sperm of irregular shape, such as mouse sperm, can be a useful mean for sorting purposes.

And the accuracy of sorting were verified by using FISH (Fluorescence In Situ Hybridization).

### MODE FOR INVENTION

From now on, examples of sex specific sperm sorting using this invention and methods to reprove will be given. However, examples described below are not limited to present invention and those skilled in the art will appreciate that various modifications, additions and substitutions are allowed without departing from the scope and spirit of the invention and it is within a technical descriptions range of present invention.

### Example 1

### The Preparation of Semen Extender

The formula of the extender used for this invention is like this.

TABLE 1
1X Salts

| Ingredients | gm/1000 ml | final Conc(mM) |
|---|---|---|
| NaCl | 5.534 | 94.59 |
| KCl | 0.356 | 4.78 |
| CaCl₂ | 0.189 | 1.71 |
| KH₂PO₄ | 0.162 | 1.19 |
| MgSO₄•7H₂O | 0.294 | 1.19 |
| NaHCO₃ | 2.106 | 25.07 |

The formula of the media for sperm motility and viability is like this.

TABLE 2
50X Metabolites

| Ingredients | mg/20 ml | 1X Conc.(mM) | |
|---|---|---|---|
| Na lactate | 2608 | 23.28 | |
| Na pyruvate | 36 | 0.33 | |
| Glucose | 1000 | 5.56 | |
| Penicillin | 62.1 | 100 | U/ml |
| Streptomycin | 50 | 50 | g/ml |

This solution was used to maintain pH.

TABLE 3
20X Hepes-NaCl

| Ingredients | gm/5 ml | gm/10 ml |
|---|---|---|
| NaCl | 0.900 | 1.8 |
| Hepes | 0.2383 | 0.476 |

### Example 2

### Sex-Dependant Sorting of Mouse Sperm

The sex-specific sorting of sperm was conducted using BD FACSAria. Mouse semen was collected and mixed with semen extender, nutrients, and buffering reagents shown in a comparative example 1. Sexually mature mouse (strain C57 BL or ICR) was inhaled with CO₂ gas and then killed by applying fracture of cervical spine. Subsequently, epididymis and seminal ducts were dissected out, submerged in 2 ml of extender, sliced into smaller pieces, and incubated in CO₂ incubator for 15-20 minutes to allow sperm to be released from the dissected tissues. Released sperm were stained with a DNA binding fluorescence dye, bisbenzimide (5 ug/ml) by using swim up method in 5% CO₂ at 34° C. for 30 minutes. Sheath pressure was adjusted to 20 psi, CO₂ was supplied by directly connecting CO₂ tank to the sheath solution tank, pH was maintained near 7.4. Sperm were stained with fluorescence dye, bisbenzimide (5 ug/ml) and illuminated with UV laser (˜340 nm) and blue laser (488 nm) simultaneously. Sperm were briefly grouped using photomultiplier (PMT) at voltage near 250 and 300 and then, gating was decided on both fluorescence data obtained from the blue laser analysis and data on nucleus size obtained from UV laser analysis. A voltage applied between two electrodes was 5000 volt.

The shape of graph was similar to the dot plot in right diagram of FIG. 7, which shows the sperm population. If the diameter of the nucleus is not used as a parameter for the distribution of dot plot was like the left graph in the FIG. 7. Dot plot was like the one in FIG. 6 when sperm's tail was removed and condition was adjusted to force to uniform the orientation (In a conventional protocol, UV laser was used to measure the whole fluorescence emitted from the head of sperm).

### Example 3

### Analysis of the Separation Efficiency of Sperm Using FISH (Fluorescence in Situ Hybridization)

To confirm that the method is working, probe #1 specific to chromosome 1 (TCT CGG CTT TGT TTT ATT TTG TTT TGG TTT) was labeled with FITC and used as a control. Probe #2, specific to chromosome Y, (TAC CCA AAC TAT AAA TAT CAG CCT CAT CGG) was labeled with Cy3 and used to detect sperm containing chromosome Y. About 200000 of sex-sorted sperm were transferred to 1.5 ml tube containing 200 ul of buffer solution (0.1M Tris-HCl). Sperms were incubated for 10 minutes and washed with 2×SCC buffer for 5 minutes twice. To de-condense over-concentrated nucleus, 200 ul of dithiothreitol (DTT, 2 mM) solution (pH 7.4) was added and the mixture incubated for 15 minutes at 37° C. After that sperm were washed twice with 2×SSC buffer and dried at room temperature. Subsequently, hybridization solution (28 ul), FITC-probe (2 ul), Cy3-probe (2 ul), and distilled water (4 ul) were added to visualize particular fluorescence. The mixture were warmed to 73° C. for 10 minutes to disintegrate double strand DNA and further incubated at 75° C. for 10 minutes to allow fluorescence-conjugated probes bind to the target sequences. Unbound and weakly-bound probes were removed by washing for 2 minutes with 2×SSC solution at 37° C. Finally, prepared sperm samples were analyzed for their fluorescence by using flow cytometry. FITC fluorescence was passed through 530/30 nm band filter. Cy3 was analyzed for its fluorescence signaling by passing through 575/26 filter band. Additionally, about 100,000 sperms were analyzed by passing through 450/40 after noise canceling.

The results are shown in FIG. 10. The ratio of X and Y-sperms were equal before sorting. However, when sperm were analyzed after sorting process, the purity was found to be higher than 90%. Although the successful sorting of mouse sperm had not been reported previously, probably, due to their hooked head, in case of this invention, purity of sorted mouse sperm were found to be quite high. In cases of this invention, this is probably due to the application of an additional parameter of width for the gating. Unlike conventional methods whereby sperm are analyzed properly, only when they are arranged in a right orientation, this invention, due to the application of additional parameter for sorting, can successfully differentiate signal from X- and Y-sperm without being affected too much by the sperm's orientation.

### Example 4

### Analysis of Sorting Accuracy by Using Embryo Fertilized with Sex-Sorted Sperm

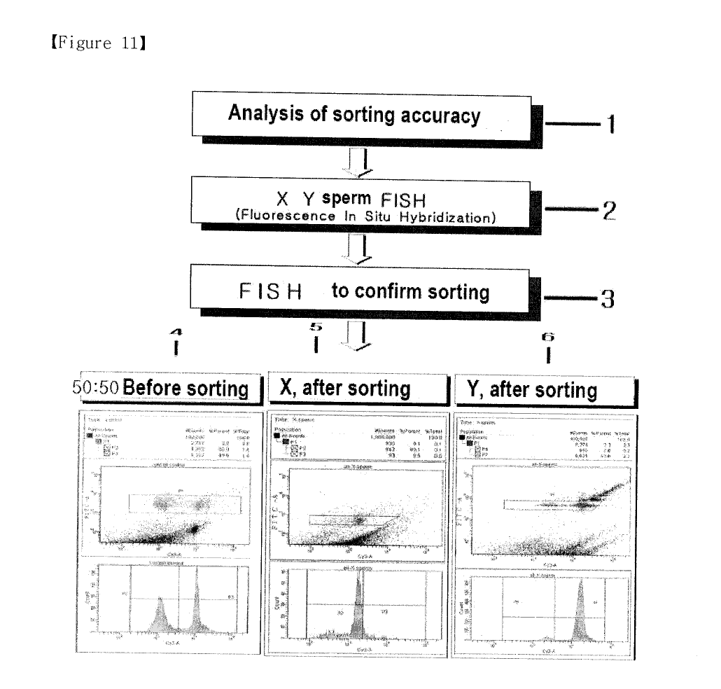

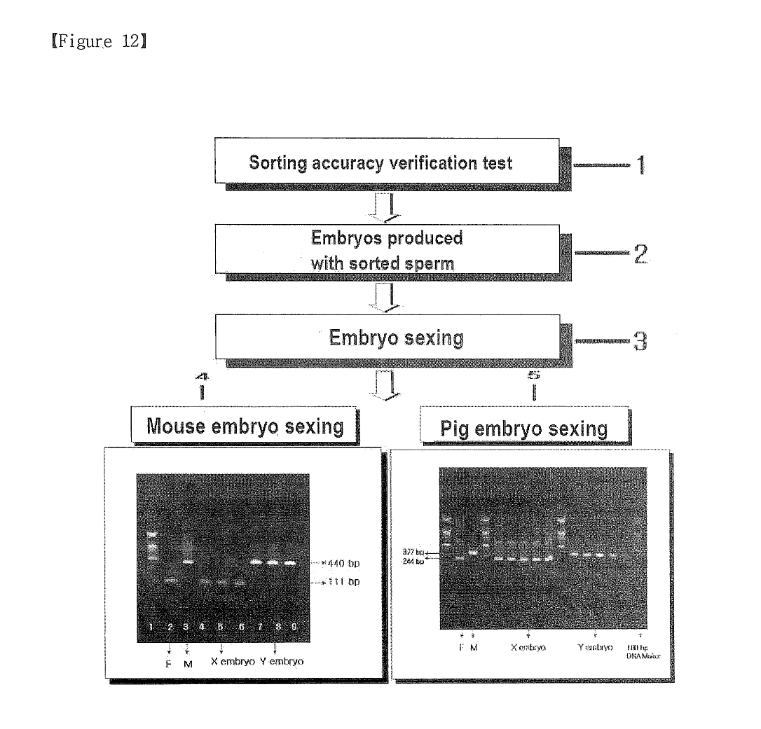

In cases of pig, sperm were handled in same way as explained above except that sperm were kept at 15 C during the process. FIG. 11 shows how the sex of embryo can be determined by using PCR amplification and electrophoresis of specific DNA sequences. Embryos fertilized with sex-sorted sperm were subjected to the nested PCR which can perform DNA amplification with very small amount of template DNA. Specific DNA sequences were amplified in the presence of 0.1 ug DNA and 0.5 unit of Taq polymerase in a 20 ul volume. First, each mouse embryo was placed in a tube and subjected to three repeat of a temperature cycle (95° C. for 2 min, 60° C. 8 minutes). Subsequently, the PCR products were used for the first amplification (30 cycles of 94° C. 3 minutes, 94° C. 1 minutes, 60° C. 1 minute and 72° C. 1 minute followed by a 72° C. 10 minutes for the completion of DNA extension). PCR products were subjected for a second amplification (35 cycles of 94° C. 3 minutes, 94° C. 5 sec., 69° C. 40 sec., and 72° C. 1 minute).

The primer sets used for the PCR amplification is shown in table 4. SRY is a Y-chromosome specific primer set and DNS is an X-chromosome specific primer set.

TABLE 4

| | Nucleotide | Base |
|---|---|---|
| Primer set | sequence | pair |
| SRY1 | AATGGGGGCCATGTCAA | 1100 |
| (sequence | | |
| No. 3) | | |
| SRY2 | TCATGAGACTGCCAACCAC | |
| (sequence | | |
| No. 4 | | |
| SRY3 | TCATGAGACTGCCAACCACAG | 440 |
| (sequence | | |
| No. 5) | | |
| SRY4 | CATGACCACCACCACCACCAA | |
| (sequence | | |
| No. 6) | | |
| DNS1 | ATGCTTGGCCAGTGTACATAG | 111 |
| (sequence | | |
| No. 7) | | |
| DNS2 | TCCGGAAAGCAGCCATTGGAGA | |
| (sequence | | |
| No. 8) | | |
| DNS3 | GAGTGCCTCATCTATACTTACAG | 244 |
| (sequence | | |
| No. 9) | | |
| DNS4 | TCTAGTTCATTGTTGATTAGTTG | |
| (sequence | C | |
| No. 10) | | |

For the purpose of sexing embryos, DNA extracted from female and male animal tissues were used as a positive control. DNA (0.1 μg) from each tissues were amplified using PCR techniques. Taq polymerase, primers (10 mol) were included in the reaction. In the case of porcine embryo, nested PCR was employed. In the first step, 3 cycles of 95° C. 2 min, 60° C. 8 min was conducted. Using the resulting products, second step of PCR amplification was conducted. For the PCR amplification, Taq polymerase of 0.5 unit and 10 pmol of primer set were mixed with resulting product (diluted 1/50000) in a total volume of 20 μl. In the first step PCR, DNA was denatured at 95° C. for 5 min, then amplified with 35 cycle of 95° C. 15 seconds, 60° C. 1 minute, and 72° C. 15 seconds, and finished with extension step of 72° C. 7 minutes. In same manner, resulting products were re-amplified with 35 cycles of 95° C. 15 seconds, 60° C. 1 minute, and 72° C. 15 seconds, and terminated with 72° C. 7 minutes.

The sequence of primer used for PCR amplification, and product sizes are like in this table.

TABLE 5

| | Nucleotide | Base |
|---|---|---|
| Primer set | sequence | pair |
| PYF | AATCCACCATACCTCATGGAC | 377 |
| (sequence No. 11) | | |
| PYR | TTTCTCCTGTATCCTCCTGC | |
| (sequence No. 12) | | |
| PCF | GTTGCACTTTCACGGACGCAG | 244 |
| (sequence No. 13) | | |
| PCR | CTAGCCCATTGCTCGCCATAG | |
| (sequence No. 14) | | |

In this invention, as shown in FIG. 11, the separation efficiency was proven to be high and the purity of sorted sperm is also very high from the test utilizing embryo sexing. These techniques specially designed to prove the accuracy of sorting sperm, confirmed to be simple and highly accurate and, therefore, can be applied to most mammalian species.

1. A method to separate X-chromosome bearing sperms and Y-chromosome bearing sperms by utilizing the difference in the diameter of nucleus inside sperm as a parameter based on the physiological characteristics of sperm.

2. The method according to claim 1, wherein the size of nucleus is measured by analyzing the gap of two time points at which sperm enter and exit the area of laser illumination.

3. The method according to claim 2, wherein, the method is utilizing the area and wave height of the wave, in accordance to time, generated when a sperm pass through laser beam as another parameter for the separation of sperms based on their physiological characteristics.

4. A method to separate X-chromosome bearing sperms and Y-chromosome bearing sperms, comprising the following steps of:
a) collecting sperm from the males of animals;
b) preparing samples by mixing sperms with extender;
c) estimating the quality of sperm based on the physiological characteristics;
d) grading the sperm into several groups based estimation;
e) separating qualified sperm into two groups, X-chromosome bearing group and Y-chromosome bearing group;
wherein the method is characterized by measuring the time taken for the sperm nucleus to pass the laser time point when sperm nucleus enter the laser and the ending point when the sperm nucleus exit the laser beam, due to the difference in the diameter of nucleus inside sperm as a parameter based on the physiological characteristics of sperm.

5. The method according to claim 4, wherein the method is characterized by staining the sperm nucleus with fluorescence dye.

6. The method according to claim 5, wherein the method is characterized by probing stained nucleus using UV laser.

7. The method according to claim 6, wherein the intensity of UV laser is lower than 20 mW.

8. The method according to claim 4, wherein the method utilizes the area and height of the wave generated when a sperm pass through laser beam as a another parameter to characterize the differences in the width of the sperm nucleus based on sperm's physiological characteristics.

9. The method according to claim 5, wherein the method further comprises the measurement of the intensity of fluorescence staining of nucleus as a parameter for the separation.

10. The method according to claim 9, wherein the method is characterized by utilizing blue laser to analyze the difference of fluorescence intensity.

11. The method according to claim 1, wherein the method further comprises utilizing the difference of forward scattering and/or side scattering as a parameter, in measuring the volume of sperm among the physiological characteristics.

12. The method according to claim 1, wherein the method is characterized by suppressing motility of sperm by putting them in hibernation temporarily under specific temperature range.

13. The method according to claim 1, wherein the method is characterized by supplying carbon dioxide to the buffer containing live sperm.

14. The method according to claim 1, wherein the method is characterized by preventing sperm aggregation and allowing continuous and homogenous supply of sperm by making the entrance of supply tube as a V-shape.

15. The method according to claim 1, wherein the method is characterized by minimizing the sperm damage caused by collision to the tube by coating the internal surface of the supply line with mineral oil or silicon.
